(19) United States
(12) Patent Application Publication
(10) Pub. No.: US 2011/0008776 A1
(43) Pub. Date: Jan. 13, 2011

(54) INTEGRATED SEPARATION AND DETECTION CARTRIDGE USING MAGNETIC PARTICLES WITH BIMODAL SIZE DISTRIBUTION

(75) Inventors: Peter Warthoe, Kobenhavn (DK); Søren Mentzel, Copenhagen S (DK); Klaus Rune Andersen, Vanlose (DK); Jens Mikkelsen, Ballerup (DK); Jacob Holst Madsen, Glostrup (DK); Per Berdén, Malmo (SE)

Correspondence Address: THE NATH LAW GROUP, 112 South West Street, Alexandria, VA, 22314, US

(73) Assignee: Atonomics A/S, Copenhagen SV (DK)

(21) Appl. No.: 12/742,520

(22) PCT Filed: Nov. 26, 2008
(86) PCT No.: PCT/EP2008/066274

§ 371 Date: Sep. 28, 2010

(30) Foreign Application Priority Data
Nov. 26, 2007 (DK) PCT/DK2007/000517
Nov. 26, 2007 (DK) PCT/DK2007/000519

(51) Int. Cl. C12Q 1/68; G01N 33/573; G01N 33/53; C12M 1/40

(52) U.S. Cl. 435/6; 435/7.4; 435/7.92; 435/287.2

(57) ABSTRACT

The present invention relates to a device and a method for quantitative detecting of the presence or absence of a target analyte in a liquid sample, the device comprising a reaction chamber having a volume of less than 200 μl comprising an immobilization matrix capable of capturing the analyte, said immobilization matrix, preferably comprising a particulate magnetic material, having a size distribution that is at least bimodal.

### TECHNICAL FIELD

The present invention relates to a device for quantitative detecting the presence or absence of a target analyte in a liquid sample, and to uses thereof.

The invention further relates to a method for quantitative detecting the presence or absence of a target analyte in a sample consisting of less than 200 μl

### BACKGROUND

Over the years, numerous simplified test systems have been designed to rapidly detect the presence of a target analyte of interest in biological, environmental and industrial fluids. In one of their simplest forms, these assay systems and devices usually involve the combination of a test reagent which is reacting with the target analyte to give a visual response and an absorbent paper or membrane through which the test reagents flow.

The contact may be accomplished in a variety of ways. Most commonly, an aqueous sample is allowed to traverse a porous or absorbent member, such as porous polyethylene or polypropylene or membranes by capillarity through the portion of the porous or absorbent member containing the test reagents. In other cases, the test reagents are pre-mixed outside the test device and then added to the absorbent member of the device to ultimately generate a signal.

Many commercially available devices and assay systems also involve a wash step in which the immune absorbing zone is washed free of non specifically bound signal generator so that the presence or amount of target analyte in the sample can be determined by examining the porous member for a signal at the appropriate zone.

In addition to the limitations of the assay devices and systems of the prior art, including the limitations of using absorbent membranes as carriers for sample and reagents, assay devices generally involve numerous steps, including critical pipetting steps which must be performed by relatively skilled users in laboratory settings. Accordingly, there is a need for one step assay devices and systems, which, in addition to controlling the flow of reagents in the device, control the timing of the flow of reagents at specific chambers in the device. In addition, there is a need for assay devices which do not require critical pipetting steps and are performing in a full quantitative way.

Today most target analyte are measured using large equipment (immune analyzers) located at central laboratories. One of the major reasons for this is that no small hand-held instrument exist today that can fulfill the critical parameters for a highly sensitive, reproducible and quantitative immune as well as DNA assay.

Accordingly, an object of the present invention was to develop a handheld device and a method capable of reliably and efficiently detecting the presence or absence of target analytes in small samples of less than 200 μl.

One major concern when quantitatively detecting presence or absence of analytes in small samples is the elimination or reduction of background signal, which disturbs the reliability and reproducibility of detecting small amounts of analyte.

Accordingly another object of the present invention was to develop a device and a method for quantitatively detecting the presence or absence of a target analyte in a small liquid sample, wherein the background unspecific signal is reduced or eliminated.

### DISCLOSURE OF THE INVENTION

In the experimental development leading to the present invention the inventors found that critical parameters for obtaining a highly sensitive, reproducible and full quantitative assay for quantitatively detecting presence or absence of analytes in small samples are to increase the signal to noise ratio by lowing the background noise. Further, efficient mixing procedures between the target analyte and tracer/capture antibodies are preferred, as well as efficient washing procedures for lowing background noise. Even further it was found that a large reaction surface between target analyte and tracer/capture antibodies is preferred. Further preferred features are efficient amplification reagent such as HRP or ALP enzyme conjugated tracer antibodies and the possibility of using temperature controlled assays.

By combining microfluid and magnetic particle technology in a special constellation the present inventors found that it was possible to fulfill the critical parameters and at the same way obtaining a relative small handheld instrument (below 500 gram), capable of analysing samples of less than 200 μl.

Surprisingly, it was found that using a bimodal size distribution of the magnetic beads (bmsMB), as opposed to the conventional single modal size distribution (smsMB) increased the signal to noise ratio. Further investigations showed that the bmsMB gives better assay performances compared to single modal size distribution, due to better mixing between MBs with bimodal size distribution, which results in optimal washing of the magnetic beads. This again reduces the unwanted background signal. Further, it was found that the use of a mixture of large and small magnetic beads results in excellent results in terms of analyte capture by obtaining a large reaction surface (the benefit of the small particle size) combined with an efficient capture and transfer of the particles which is the benefit of the large magnetic particles. Thus, it was found that by using magnetic particles with a bimodal size distribution both high signal, low noise and excellent mobility can be obtained.

Accordingly, in a preferred aspect of the invention it relates to a device for quantitative detecting the presence or absence of a target analyte in a liquid sample having a volume of less than 200 μl, the device comprising a reaction chamber comprising an immobilisation matrix capable of capturing the analyte, said immobilisation matrix comprising magnetic material having a size distribution that is at least bimodal.

In a preferred aspect the invention relates to a device for quantitative detecting the presence or absence of a target analyte in a liquid sample, the device comprising a reaction chamber in the form of a capillary channel having a volume of less than 200 μl, the reaction chamber comprising:
a. a first part (3) comprising a reaction chamber and a sample inlet for the introduction of a sample containing an analyte the first part further comprising an immobilisation matrix comprising magnetic material having a size distribution that is at least bimodal;
b. a second part (5 and 6) comprising means for detection of the target analyte,
c. a solution inlet (8) for introduction of washing solutions and reaction mixtures;
d. means for transferring an immobilised analyte from the first part to the second part of the chamber and vice versa; and
e. a discharge outlet for the discharge of waste products;

where the first and second parts are separated such that liquid sample material from the first part of the chamber may not enter the second part of the chamber.

In a further aspect the invention relates to the use of a device according to the invention for the quantitative detection of the presence or absence of a target analyte in a sample.

In a further aspect the invention relates to a method for quantitative detecting the presence or absence of a target analyte in a sample consisting of less than 200 μl liquid, comprising the steps of:
a) providing an analyte containing liquid sample consisting of less than 200 μl liquid;
b) supplying the liquid sample to reaction chamber,
c) contacting the sample in the reaction chamber with an immobilisation matrix capable of capturing the analyte, said immobilisation matrix comprising magnetic material having a size distribution that is at least bimodal;
d) immobilising the immobilisation matrix comprising the captured analyte;
e) washing the immobilisation matrix comprising the captured analyte with a washing solution;
f) transferring the immobilisation matrix comprising the captured analyte to the detector part of the chamber; and
g) detecting the presence or absence of a target analyte using conventional detection means.

In a further aspect the invention relates to a kit of parts comprising a device according to the invention and a magnetic material.

### BRIEF DESCRIPTION OF THE DRAWINGS

The invention is explained in detail below with reference to the drawings, in which

FIG. 1 illustrates a schematic presentation of a sample device comprising a microfluid channel having a first part (3) and a second part (5, 6), an application zone (1), a separation chamber (2), a first capillary channel (3), a collection chamber (4a), a waste outlet (4b), a washing chamber (5), a detection chamber (6), magnetic particles (having a bimodal size distribution) (7) (which may be transferred between the first and the second part) located in washing chamber, an inlet channel for washing and detector solution (8), a physical barrier (10 (vertical), 10′ (incline)) between the separation chamber and the first capillary channel, capillary micro channels (11) in the first capillary channel (3), corona treatment (12) (symbolised by the grey shade) of the first capillary channel, and a detector unit (14). When starting the assay the magnetic particles are situated in the first part (3).

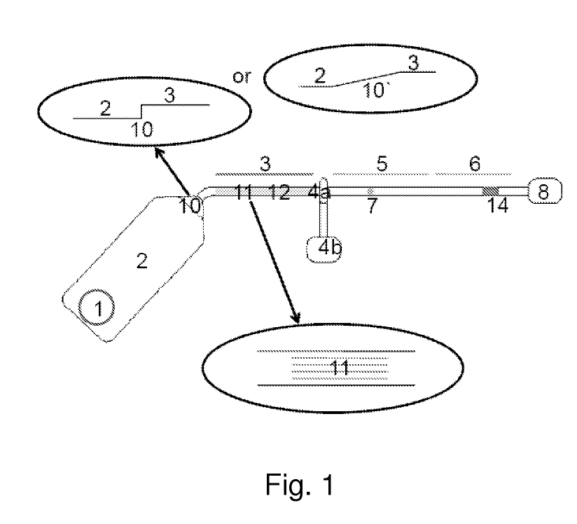

FIG. 2 illustrates the same principle as in FIG. 1 with a three dimension illustration.

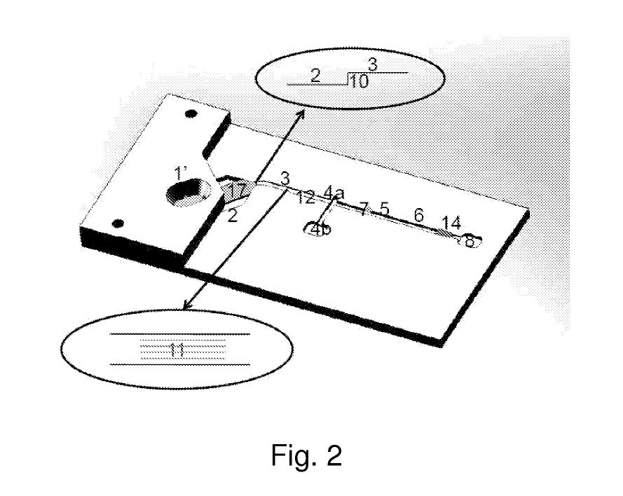

FIG. 3 illustrates a schematic site view of a separation device comprising a microfluid channel (3), an application well (1′), a separation chamber (2), a first capillary channel (3), a physical barrier (10′) between the separation chamber and the first capillary channel, a hydrophilic filter material (17), and a prefilter (15).

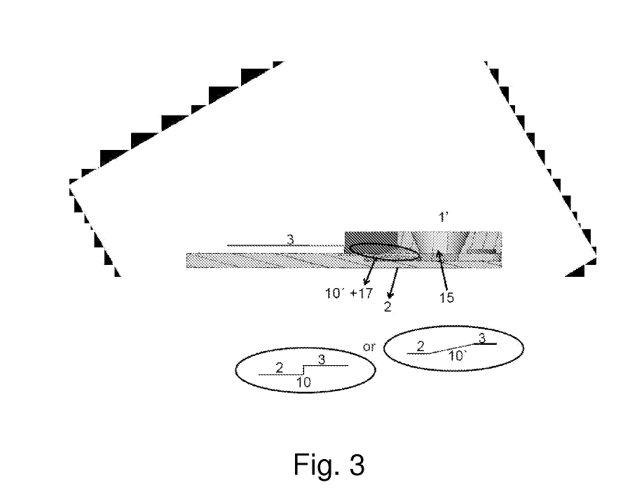

FIG. 4a illustrates a schematic site view of an integrated separation and detection device comprising a microfluid channel (3,5,6), an application well (1), a separation chamber (2) and a hydrophilic filter (17), a first capillary channel (3), serum/plasma (18) in the first capillary channel, signal solution (19) in washing (5) and detector chamber (6), light trap version A (20) in connecting junction between the first capillary channel (3) and the washing chamber (5), and a detector unit (14).

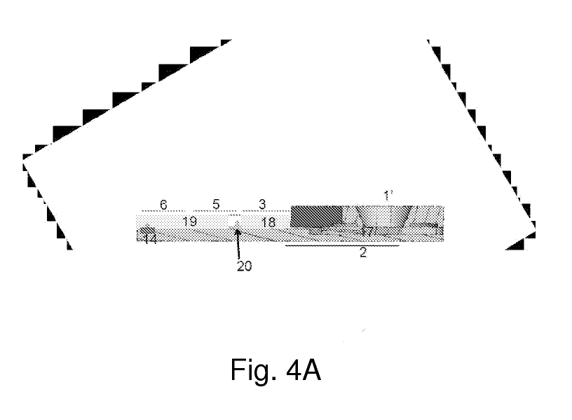

FIG. 4b illustrates a schematic site view of an integrated separation and detection device comprising a microfluid channel (3,5,6), a application well (1), a separation chamber (2) and a hydrophilic filter (17), a first capillary channel (3), serum/plasma (18) in the first capillary channel, signal solution (19) in washing (5) and detector chamber (6), a light trap version B (20′) (e.g. by introducing a bend on the path from the first part to the second part of the chamber, so the exit point from the first part and the entry point of the second part in different levels) in connecting junction between the first capillary channel (3) and the washing chamber (5), and a detector unit (14).

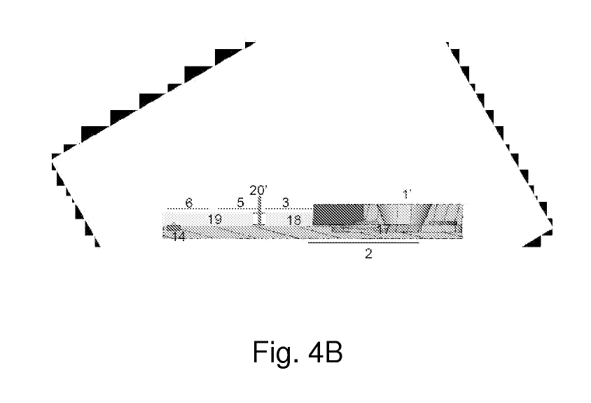

FIG. 5 illustrates same principle as in FIG. 1 with a three dimension illustration including more features. A integrated separation and detection device comprising a microfluid channel having three compartements (3, 5, 6), an application well (1′), a separation chamber (2), a first capillary channel (3), a collection chamber (4) with a waste outlet, a washing chamber (5), a detection chamber (6), magnetic particles location in washing chamber (7), an inlet channel for washing and detector solution (8), a physical barrier (10, 10′) between the separation chamber and the first capillary channel, capillary micro channels (11) in the first capillary channel (3), a detector unit (14), a first compartment for detection solution A (9), a second compartment for detection solution B (15), a washing solution compartment (16), and a blood lid (12a).

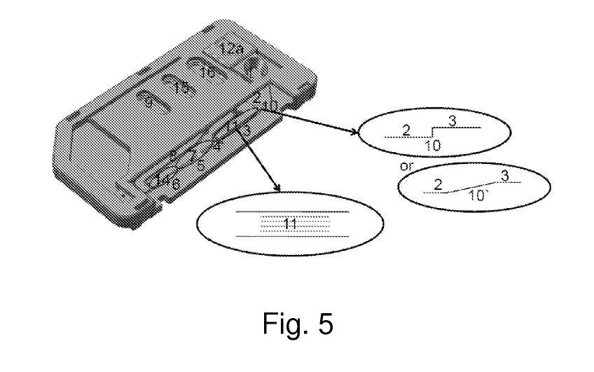

FIG. 6 illustrates a top view of an integrated separation and detection device comprising an application well (1), a filtration area (2), a plasma inlet (21), a first part channel (3) connected to the absorbing barrier and capillary stop (22). A blister container with washing solution (23) is connected to the microfluid system via channel (24) connected to channel (25) and into the detection area via channel (26) and (6). The washing channel (5) ends in the collection chamber (at the capillary stop (22)), where it is connected to two side channels (27), which end in a waste container (not shown). In the washing channel, there is a detection area (window) (6, 14). Blister (28) is connected to channel (30) and blister (29) is connected to channel (31). The channels (30) and (31) are connected to channel (32), which is connected to channel (33), when signal solutions from channel (30) and (31) reach channel (33), the remaining signal solutions enter channel (34) and are mixed in channel (35), which is connected to the plasma channel at point (26).

FIG. 7 illustrates a schematic top view of the area of the capillary stop (22) and the two side channels (27) as described in FIG. 6.

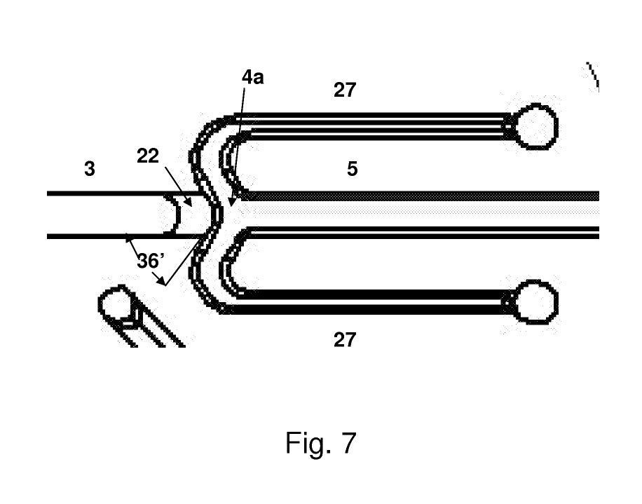

FIG. 8 illustrates sensor data for the measurement of 0 pg/ml-16,000 pg/ml BNP (by use of the assay according to example 1). “New PMT” is the PMT referred to in the example.

### DEFINITIONS

In the context of the present invention, by “capillary channel” is meant a narrow tube or channel through which a fluid can pass. Preferably the diameter of a capillary channel according to the invention is less than 10 mm. Even more preferred the diameter of a capillary channel according to the invention is less than 5 mm, such as less than 4 mm, or less than 3 mm or even less than 2 mm. In a most preferred aspect the capillary channel has a diameter of 1 mm or less.

In the context of the present invention the term “unimodal” has the conventional mathematical meaning of unimodality i.e. distributions having only one mode. A function f(x) between two ordered sets is unimodal, if for some value m (the mode) it is monotonically increasing for x≦m and monotonically decreasing for x≧m. In that case, the maximum value of f(x) is f(m) and there are no other local maxima.

In the context of the present invention the term “bimodal” has the conventional mathematical meaning of bimodality, i.e. distributions having two modes. Generally, bimodal distributions are a mixture of two different unimodal distributions.

### DETAILED DESCRIPTION OF THE INVENTION

Signal detection in microfluidic systems is often jeopardised by a very low sensitivity requiring large amounts of analyte to generate a reliable and reproducible signal. Much effort has been put into development of more sensitive and sophisticated detection means. Surprisingly, less has, however, been done in order to remove or reduce the level of unspecific signal (noise). The present inventors surprisingly found that simple measures reducing the noise of the system improved the reproducibility and the sensitivity of the system significantly.

The inventive concept of the present invention may be seen in general as the physical separation, in a microfluidic system, of the steps of binding, immobilising and washing an analyte and the steps of detecting the analyte. Preferably, any signal deriving from non-analyte species (background signal) remains in the first part (3) of the device (or the first steps in the method), whereas in the second part of the device (subsequent steps in the method) the signal derived from the analyte, with a minimal background signal, is detected.

It was surprisingly observed that when using magnetic particles having a non-unimodal size distribution, such as a bimodal size distribution, a more efficient performance in terms of washing efficiency and time was obtained. Presumably, a more efficient binding of analyte combined with a more efficient capture and mobility of particles were obtained when using magnetic particles having mixed size distributions. Accordingly, in a preferred aspect of the invention the immobilisation matrix has an at least bimodal size distribution. In another aspect of the invention the immobilisation matrix has a trimodal size distribution.

The invention thus relates to a device for quantitatively detecting the presence or absence of a target analyte in a liquid sample having a volume of less than 200 μl, the device comprising a reaction chamber comprising an immobilisation matrix capable of capturing the analyte, said immobilisation matrix having a size distribution that is at least bimodal.

In another embodiment the size distribution is trimodal.

Preferably the immobilisation matrix comprises magnetic material

Preferably the size distribution of the immobilisation matrix is bimodal with one population of particles having a mean diameter of below 2 μm, such as a diameter of or below 1.5 μm or such as a diameter of or below 1.0 μm, and another population of magnetic particles having a mean diameter of above 2 μm, such as 2.5 μm or above or 2.8 μm or above or 3.0 μm or above, or even 5.0 μm or above.

The inventive concept of the present invention may be seen in general as the physical separation, in a microfluidic system, of the steps of binding and immobilising an analyte and the steps of detecting the analyte. Preferably, any signal deriving from non-analyte species (background signal) remains in the first part (3) of the device (or the first steps in the method), whereas in the second part of the device (later steps in the method) the signal derived from the analyte, with a minimal background signal, is detected. Accordingly, in one aspect the invention relates to a device for quantitative detecting the presence or absence of a target analyte in a liquid sample, having a volume of less than 200 μl, the device comprising a reaction chamber in the form of one or more capillary channels the reaction chamber comprising:
a. a first part (3) comprising a capillary channel having a volume of less than 200 μl, l a sample inlet (1) for the introduction of a sample containing an analyte, and a discharge outlet (4b) for the discharge of waste products;
b. a second part (5) comprising means for detection (14) of the target analyte, and a solution inlet (8) for introduction of washing solutions and reaction mixtures; and
c. means for transferring an immobilised analyte from the first part to the second part of the chamber and vice versa;

where the first and second parts are separated such that other liquid sample material may not enter the second part of the chamber. By other sample material is meant sample material excluding the analyte.

In one other aspect the invention relates to a device for quantitative detecting the presence or absence of a target analyte in a liquid sample, having a volume of less than 200 μl, the device comprising
a. a first part (3) comprising a reaction chamber and a sample inlet for the introduction of a sample containing an analyte the first part further comprising an immobilisation matrix comprising magnetic material having a size distribution that is at least bimodal;
b. a second part comprising means for detection of the target analyte,
c. a solution inlet (8) for introduction of washing solutions and reaction mixtures;

d. means for transferring an immobilised analyte from the first part (3) to the second part (5) of the chamber and vice versa; and
e. a discharge outlet (4b) for the discharge of waste products;

where the first and second parts are separated such that liquid sample material from the first part of the chamber may not enter the second part of the chamber.

The reaction chamber may contain several compartments or parts. Further each part may be divided into further parts or compartements wherein specific reactions are to occur. By separating the reaction chamber in a first part (3) for binding the analyte and a second part (5) and detecting the analyte, a significant reduction in background signal could be obtained.

In a preferred aspect the sample to be analysed preferably has a volume of less than 200 μl. In an even more preferred aspect the sample to be analysed has a volume of less than 150 μl, even more preferred less than 100 ηl, even more preferred less than 90 μl, such as less than 80 μl, less than 70 μl, or even less than 60 μ. In an even more preferred aspect the sample to be analysed has a volume of less than 50 μl, even more preferred less than 45 μ1, even more preferred less than 40 μl, such as less than 30 μ, less than 30 μor even less than 25 ρl.

In a preferred aspect the first part (3) of the capillary channel has a volume of less than 100 μl. In an even more preferred aspect the first part of the capillary channel has a volume of less than 90 μl, even more preferred less than 80 μl, even more preferred less than 70 μl, such as less than 60 μl, less than 50 μl or even less than 40 μl. In an even more preferred aspect the first part of the capillary channel has a volume of less than 30 μ, even more preferred less than 25 μ, even more preferred less than 20 μl, such as less than 15 μl, less than 10 μl or even less than 5 μl. The same preferred volumes apply for the second part of the reaction chamber. The reaction chamber comprises a first (3) and a second part (5). In a preferred aspect both the first and the second part are made of capillary channels. The first and second part may be separated e.g. by a collection chamber from which residual sample matter and added reagents may be collected and later expelled. Such a collection chamber, and the volume thereof is not to be understood as part of the reaction chamber or the preferred volumes thereof.

In a preferred aspect of the invention the means for transferring the immobilised analyte from the first part to the second part of the chamber and vice versa is an external magnetic force generating source, which can apply a magnetic field to the chamber and be moved along the edge of the chamber on demand.

In one aspect the first part of the capillary channel is connected to a filter mechanism integrated into the device. The inlet of sample (e.g. serum or plasma) preferably comes through the filter device.

In one aspect of the invention the first and second parts are separated by a collection chamber (4a). The collection chamber may serve the purpose of separating the first and second parts such that liquid sample material, other then analyte species actively transported between the first and second part, may not enter the second part of the chamber. The collection chamber also serves the purpose of an outlet for waste products such as washing solution and residual sample material. The placement of the collection chamber between the first and the second part provides that the collection chamber serves as an outlet for material from both the first and the second part of the chamber.

In a preferred aspect of the invention, in order to move magnetic particles comprising the immobilised analyte most efficiently, a magnetic field is moved along the top edge (3, 5, 6) of the chamber on demand.

In a preferred aspect of the invention the first and second parts are separated such that a significant part of the signal (e.g. light) may not be transferred from the first part of the chamber to the detector part of the second part of the chamber. By a significant part is meant more than 50%, such as more than 75% or even more than 90%, or even more than 99%. This may be achieved by placing the exit point from the first part and the entry point of the second part in different levels e.g. by introducing a bend (20′) on the path from the first part to the second part of the chamber, such that signal (in the form of light rays) from the first part of the chamber may not enter the detection part of the second chamber. Another possibility is introducing a bend in the second part of the chamber such that the detector part is not in line with the entry point of the analyte to the second part of the chamber. A preferred possibility is the placement of a light-permeable barrier (20) between the two parts such that a significant part of the light is prevented from entering the second part from the first part. Of course the barrier must not prevent the transfer of analyte (e.g. via magnetic particles) from the first and second parts.

Preferably, the surface structure and the colour of the internal surface of the reaction chamber, or at least the second part of the chamber, is non-reflecting and/or light absorbing, respectively. In one aspect of the invention the non-reflecting and/or light absorbing surface is obtained by obscuring and/or darkening of the surface. In a preferred aspect the darkening is blackening. Most preferably the colour of the internal surface of the reaction chamber is black.

In a preferred aspect of the invention the means for detection of the target analyte are selected among surface acoustic wave (SAW) detectors, spectrophotometers, fluoro-meters, CCD sensor chip(s), COOS sensor chip(s), PMT detector(s), or any suitable light detector.

In a preferred aspect the internal width and height of the reaction chamber, or at least the first part (3) of the reaction chamber, is 0.1-5 mm and 0,05 -2 mm respectively. More preferably, the internal width and height of the reaction chamber, or at least the first part of the reaction chamber, is 0.25-2 mm and 0.2-1 mm, respectively

In a preferred aspect the length of the reaction chamber is 2-30 mm, more preferably 5-20 mm.

The device according to the invention may be used for the quantitative detection of the presence or absence of a target analyte in a sample. Preferably, the sample is derived from blood. In one aspect the sample is serum. In one aspect the sample is plasma. Plasma may obtained by applying an anti coagulant to the blood sample to be analysed. Preferred anti-coagulant may be selected among the group comprising K3-EDTA, citrate and heparine.

In a preferred aspect of the invention the sample is of human origin.

In one aspect the invention relates to a method for quantitative detecting the presence or absence of a target analyte in a sample consisting of less than 200 μl liquid, comprising the steps of:
a) providing an analyte containing liquid sample consisting of less than 200 ρl liquid;
b) supplying the liquid sample to reaction chamber,
c) contacting the sample in the reaction chamber with an immobilisation matrix capable of capturing the analyte, said immobilisation matrix, preferably comprising magnetic material, having a size distribution that is at least bimodal;
d) immobilising the immobilisation matrix comprising the captured analyte;
e) washing the immobilisation matrix comprising the captured analyte with a washing solution;
f) transferring the immobilisation matrix comprising the captured analyte to the detector part of the chamber; and
g) detecting the presence or absence of a target analyte using conventional detection means.

In another aspect the invention relates to a method for quantitative detecting the presence or absence of a target analyte in a sample consisting of less than 200 μl liquid, comprising the steps of:

a) providing an analyte containing liquid sample consisting of less than 200 μl liquid;
b) supplying the liquid sample to a first reaction part of a chamber, the chamber comprising a first reaction part and a second detection part, the two parts being physically separated such that liquid sample material cannot enter into contact with the second detection part;
c) contacting the sample in the first reaction part of a chamber with an immobilisation matrix having a size distribution that is at least bimodal and capable of capturing the analyte;
d) immobilising the immobilisation matrix comprising the captured analyte;
e) transferring the immobilisation matrix comprising the captured analyte to the second part of the chamber;
f) remobilising and washing the immobilisation matrix comprising the captured analyte with a washing solution;
g) immobilising the immobilisation matrix comprising the captured analyte;
h) optionally, discarding the washing solution
i) optionally, remobilising the immobilisation matrix comprising the captured analyte and repeating steps f) to h);
j) transferring the immobilisation matrix comprising the captured analyte to the detector part of the second part of the chamber; and
k) detecting the presence or absence of a target analyte using conventional detection means.

By separating the steps a)-d) of binding the analyte in one compartment and the steps e)-k) of washing and detecting the analyte in a second compartment a significant reduction in background signal was observed.

In a preferred aspect the method further comprises a step a′) of contacting the analyte with a biological marker capable of binding to the analyte. The biological marker may be an antibody e.g. with enzyme horseradish peroxidise (HRP), biotin or alkaline phosphatase (ALP). Thereby, the analyte may become more detectable by increasing the signal for detection. In a preferred aspect of the method according to the invention the step a′) of contacting the analyte with a biological marker, capable of binding to the analyte is performed prior to step e). Thereby, the presence of unbound biological marker in the detection part of the method is minimised and the background signal is significantly reduced. In a preferred aspect of the invention the biological marker is capable of reaction with a substrate whereby signal may be amplified. Accordingly, in one aspect of the invention the method further comprises a step f′) of contacting the immobilisation matrix comprising the captured analyte with a substance capable of reacting with the biological marker.

In a preferred aspect of the invention the biological marker is one [or more] selected from compounds, mono-, oligo- and polyclonal antibodies, antigens, receptors, ligands, enzymes, proteins, peptides and nucleic acids. Preferably, the biological marker is one or more selected from the group having the properties of light absorption, fluorescence emission, phosphorescence emission, or luminescence emission.

In a preferred aspect the immobilisation matrix comprises magnetic material. In a preferred aspect the step e) is performed by moving a magnetic source along the external edge of the first reaction chamber toward the second detection chamber.

The magnetic material is preferably selected from the group comprising magnetic particles, magnetic nanoparticles and superparamagnetic nanoparticles.

In a preferred aspect of the invention the conventional detection means are selected among surface acoustic wave (SAW) detectors, spectrophotometers, fluorometers, CCD sensor chip(s), COOS sensor chip(s), PMT detector(s), or any suitable light detector.

The method according to the invention may be used for the quantitiative detection of the presence or absence of a target analyte in a sample. Preferably, the sample is derived from blood. In one aspect the sample is serum. In one aspect the sample is plasma. Plasma may obtained by applying an anti coagulant to the blood sample to be analysed. Preferred anti-coagulant may be selected among the group comprising K3-EDTA, citrate and heparine. In a preferred aspect of the invention the sample is of human origin.

In one aspect, the invention relates to a kit of parts comprising a device as defined above and a magnetic material according to the invention. Preferably this kit is for use in detection of the presence or absence of a target analyte in a sample.

### Examples

### Example 1

An Assay Cycle in the Integrated Separation and Detection Device

The purpose of this example was to illustrate
1. The measuring principle with the analyte Brain Natriuretic Peptide (BNP) as example
2. The detection limit
3. The detection range
4. The CV values at different BNP concentrations
5. Measuring of BNP in blood samples

Materials

Standards: Range 0 pg/ml-16,000 pg/ml BNP was measured by use of the method in this example.

Samples: 4 different blood samples from healthy volunteers and 4 different samples from patients with heart failure were measured by use of the method in this example.

Antibodies: Magnetic particles (MP) coated with BNP monoclonal catching antibody. Tracer antibody is a HRP label monoclonal BNP antibody. Tracer antibody was placed directly in the blood separation filter.

Blood stabilizing reagent: EDTA is added to either the capillary channel or the blood sample.

Washing solution: TBS+0.05 wt·vol % Twen and 0.05 wt·vol % BSA

Detector solution: Pierce SuperSignal ELISA Femto Maximum Sensitivity Substrate (composed of 1 vol-part signal solution from blister A and 1 vol-part signal solution from blister B according to step 17 below)

Detector: PMT detector (Hamamatsu)

Assay temperature: 19° C.

Mechanics and Electronics: All mechanical parts, electronics controllers and software are produced in-house by the assignee company.

Assay procedures:

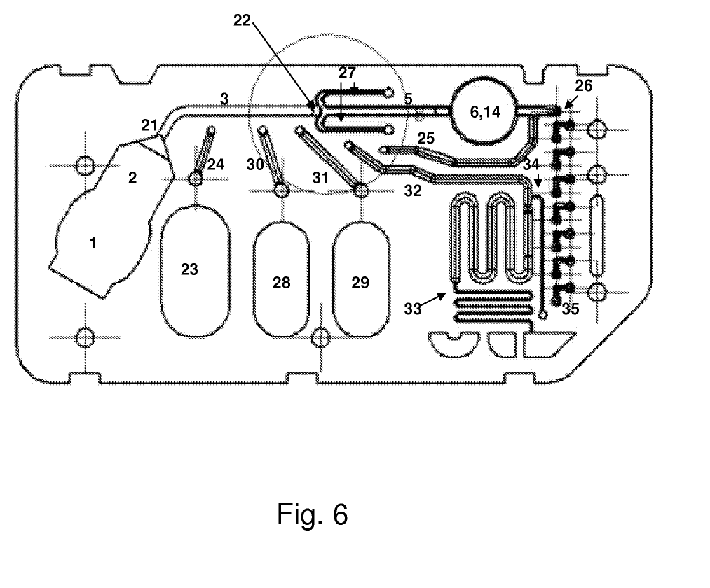

(using a separation and detection device as illustrated at FIG. 6)
1. 36-50 μl sample or standard was applied to the filtration area (2)
2. After separation 4.6 μl plasma entered the plasma channel via the plasma inlet (21), capillary forces drag the sample into the reaction chamber).
3. Plasma enters the plasma channel (3) and runs up to the light absorbing barrier and capillary stop (22)
4. In the plasma channel (which is coated with magnetic particles) the magnetic particles dissolved into the plasma entering the plasma channel (3)
5. The MPs are moved slowly backwards/forwards in the plasma channel (3) during assay incubation time using an external magnet drive mechanism.
6. After assay incubation time, all the MPs are concentrated and fixed via external magnet drive mechanism near the capillary stop location (22).
7. Blister with washing solution (23) is punctured and the washing solution enters the microfluid system via channel (24) connected to channel (25) and into detection area via (26) and (6).
8. The washing solution flows further via washing channel (5) until the washing solution arrives at the capillary stop (22) where it contacts the plasma front and proceeds directly via the collection chamber with side channels (27) into waste container (not shown).
9. The MPs are moved via the capillary stop (22) barrier into the washing channel (5) using an external magnet drive mechanism.
10. The MPs are moved slowly backwards/forwards in the washing channel (5) using an external magnet drive mechanism.
11. The MPs are concentrated and fixed via external magnet drive mechanism in the middle of the washing channel (5).
12. More washing solution is injected via the washing solution containing blister (23).
13. Due to higher pressure (compare to plasma channel) in the collection chamber and side channels (27) the newly injected washing solution will enter the lower pressured plasma channel (3) thereby pushing the plasma further backwards into the blood filtration area (2).
14. Further washing cycles may be performed by repeating step 10 and 11.
15. The external magnet drive mechanism moves the MP into the detection area (window) (6, 14) where the MPs are fixed above the centre of the detection window (6, 14).
16. The wash solution is replaced with light generation solution in blister (28) and (29) in the following way:
17. Signal solution blister A (28) and signal solution blister B (29) are mixed 1:1 via channel (30) connected to channel (31) into (32).
18. Via channel (32) the first 60 uL mixed solution fills up the channel (33).
19. When pressure increases at the end of channel (33) the signal (light) generating solution enters the mixing unit via channel (34).
20. The two solutions are mixed via the mixing unit (35).
21. After 7 mixing cycles in three dimensions (x,y,z) mixing unit, the signal (light) generating solution enters the detection area (6, 14) and proceeds further into the washing channel (5) and arrives at the capillary stop (22) where is reaches the plasma front that has been exchanged with washing solution due to pressure difference between the symmetric waste channel (27) and the plasma channel (3) see step 13.
22. The external magnet drive mechanism fixing the MPs above the centre of the detection area (step 15) is quickly moved towards to filtration area (2), thereby realising the MPs over the detection window (6, 14).
23. The PMT detector is counting the light coming from the MPs via photon counting.

Results

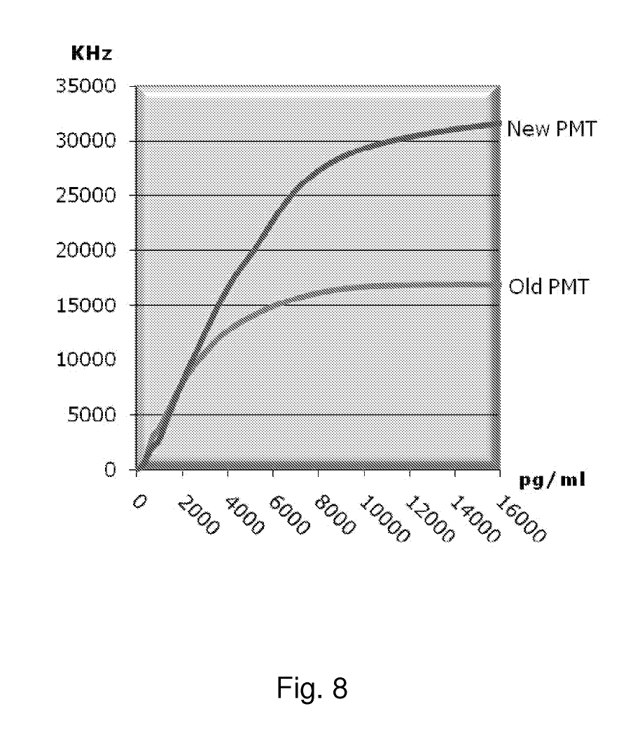

The standard curve shows linearity for the range 0-2000 pg/ml with a reasonable measuring range at 0-10,000 pg/ml (FIG. 8).

Expectedly, the results of the blood samples from healthy volunteers and the heart failure patients show that the BNP concentrations of the healthy volunteers are in the low end of the range and the BNP concentrations of the patients are 5-10 times higher. The CV values are satisfactory low.

TABLE 1
Results Measurement of Whole Blood Samples

| Samples | BNP Concentration | | CV Value |
|---|---|---|---|
| Zero plasma sample | 0 | pg/mL | 13% |
| 4 patient whole blood | 16-17 | pg/mL | 12% |
| samples | | | |
| 4 spiked whole blood patient | 96-145 | pg/mL | 10% |
| samples | | | |

Conclusion

The results show that the following key performance characteristics for the separation and detection device were accomplished:
Lower detection limit: below 5 pg/ml
Measuring range: 0 to 10,000 pg/ml
Precision: CV below 5% in the medium/high range and below 15% at the low end
Turn-Around-Time: below 15 min.
Sample materials:
   Human whole blood, optionally taken directly from a finger tip
   EDTA stabilized blood
   Plasma isolated via centrifugation

Based on the example above, it can be concluded that it is possible to detect the analyte BNP in concentration as low as the sub 5 pg/ml area with acceptable CV values and total spanning over a detection range at <5 pg/ml to >10,000 pg/ml with a linear range in the range 0-2000 pg/ml.

### Example 2

### Coating the Capillary Channel of the Device with a Hydrophilic Substance

Magnetic Particles (MP) 1 μm or 2.8 μm in diameter labelled with antibodies interacting with antigen (analyte) were stored in a stabilizing water solution with low surface tension. The MP was mixed with a sucrose solution to hold a final content of 5 wt·vol %. A typical MP concentration in the final solution for dispensing is 6 ng/ml.)

Dispensing of Magnetic Particles:

A capillary channel was washed ultrasonically in a 50vol % water solution of 2-propanol and corona treated 25 W/2 s to increase the hydrofilicity prior to dispensing. The prepared magnetic particles were dispensed into the capillary channel using an automatic high precision dispensing instrument (Nanodrop NS-1 Stage). A total volume of 1 μl was dispensed along the channel, as 4 drops of 250 nl. The pattern and volume of the dispensing may be adjusted so that the channel surface is covered but the integrity of the capillary stop is intact.

Drying and Storage:

The device comprising the capillary channel was placed horizontally for 3-5 minutes at room temperature to allow the liquid coating to evaporate from the capillary channel leaving the magnetic particles and the sucrose, thereby producing a layer of protected and easily soluble MP at the bottom of the capillary channel.

The prepared cartridge is finally stored at 4-8° C. in a sealed aluminium foil bag with silica to achieve good long term stability.

It was observed that the device comprising the capillary channel treated with the sucrose solution and stored, would fill much faster (approx. 3 times) with sucrose treatment than without. Further, a more reproducible final detection assay was obtained.

### Example 3

### Bimodal Size Distribution of Magnetic Particles

The signal/background ratio using bimodal size distribution of the magnetic particles (bmsMP) compared to single modal size distribution (smsMP) was tested in a BNP assay.

Method:

Preparation of smsMPs

Streptavidin magnetic particles (2.8 μm Dynal M280) with biotinylated monoclonal mouse anti-human antibody specific to C-terminal portion of BNP were prepared in a final concentration of 6 ng/ml in a final solution of 5 wt·vol % sucrose. The magnetic particle suspension was kept in a 0.2 ml PCR tube and was mixed just prior to dispensing.

Preparation of bmsMPs

Streptavidin magnetic particles (2.8 μm Dynal M280 and 1 μm Seramac) with biotinylated monoclonal mouse anti-human antibody specific to C-terminal portion of BNP were mixed 1:1 to a final concentration of 6 ng/ml in a final solution of 5 wt·vol % sucrose. The magnetic particle suspension was kept in a 0.2 ml PCR tube and was mixed just prior to dispensing.

The MP was dispensed in the capillary channel as described in example 2.

Table 2 shows the difference between BNP assays run using one size magnetic particle distribution compared to bimodal magnetic particles size distribution.

TABLE 2
Assay comparisons between smsMPs and bmsMPs

| | signal/ | signal/ |
|---|---|---|
| BNP | background - | background - |
| concentration | smsMP | bmsMP |
| 0 pg/ml | 0x | 0x |
| 4 pg/ml | 1.5x | 3.3x |
| 25 pg/ml | 23x | 48x |
| 300 pg/ml | 832x | 1347x |
| 1000 pg/ml | 1938x | 3298x |

The results show that significant better signal/background ratio can be obtain using bimodal magnetic particles size distribution in the BNP assay.

Further, the reproducibility of the assay (good reproducibility result in a low % CV) using bimodal size distribution of the magnetic particles (bmsMP) compared to single modal size distribution (smsMP) was tested in the BNP assay.

Table 3 shows the difference of reproducibility between BNP assays run using one size magnetic particle distribution compared to bimodal magnetic particles size distribution.

TABLE 3
Assay comparisons between smsMPs and bmsMPs

| BNP | % CV - smsMP | % CV - bmsMP |
|---|---|---|
| concentration | (n = 10) | (n = 10) |
| 0 pg/ml | 18 | 13 |
| 4 pg/ml | 16 | 12 |
| 25 pg/ml | 15 | 11 |
| 300 pg/ml | 7 | 7 |
| 1000 pg/ml | 5 | 3 |

It can be seen that significant better % CV values can be obtained using bimodal magnetic particles size distribution in the BNP assay.

1-19. (canceled)

20. A device for quantitative detecting the presence or absence of a target analyte in a liquid sample having a volume of less than 200 μl, the device comprising a reaction chamber comprising an immobilization matrix capable of capturing the analyte, said immobilization matrix having a size distribution that is at least bimodal, wherein the immobilization matrix comprises magnetic material, where the magnetic material is selected from the group comprising magnetic particles, magnetic nanoparticles and superparamagnetic nanoparticles.

21. A device according to claim 20 comprising:
a. a first part comprising a reaction chamber (3) and a sample inlet for the introduction of a sample containing an analyte;
b. a second part (6) comprising means for detection of the target analyte,
c. a solution inlet (8) for introduction of washing solutions and reaction mixtures;
d. means for transferring an immobilized analyte from the first part (3) to the second part (5 and 6) of the chamber and vice versa; and
e. a discharge outlet (4b) for the discharge of waste products;
where the first and second parts are separated such that liquid sample material from the first part of the chamber may not enter the second part of the chamber.

22. A device according to claim 21, where the first and second parts are separated by a collection chamber (4a).

23. A device according to claim 20 wherein the first and second parts are separated such that light may not be transferred from the first part of the chamber to the detector part of the second part of the chamber.

24. A device according to claim 20, wherein a surface structure and the colour of the internal surface of the reaction chamber is non-reflecting and/or light absorbing, respectively.

25. A device according to claim 21, wherein the means for detection of the target analyte are selected among surface acoustic wave (SAW) detectors, spectrophotometers, fluorometers, CCD sensor chip(s), COOS sensor chip(s), PMT detector(s), or any suitable light detector.

26. A method for the quantitative detection of the presence or absence of a target analyte in a sample comprising the device according to claim 20.

27. Method for quantitative detecting the presence or absence of a target analyte in a sample consisting of less than 200 μl liquid, comprising the steps of:
a) providing an analyte containing liquid sample consisting of less than 200 μl liquid;
b) supplying the liquid sample to reaction chamber,
c) contacting the sample in the reaction chamber with an immobilization matrix capable of capturing the analyte, said immobilization matrix comprising magnetic material having a size distribution that is at least bimodal, where the magnetic material is selected from the group comprising magnetic particles, magnetic nanoparticles and superparamagnetic nanoparticles;
d) immobilizing the immobilization matrix comprising the captured analyte;
e) washing the immobilization matrix comprising the captured analyte with a washing solution;
f) transferring the immobilization matrix comprising the captured analyte to the detector part of the chamber; and
g) detecting the presence or absence of a target analyte using conventional detection means.

28. A method according to claim 27, where the immobilization matrix has a trimodal size distribution.

29. A method according claim 27, further comprising a step of contacting the analyte with a biological marker capable of binding to the analyte.

30. A method according to claim 29, where the biological marker is an antibody e.g. coupled with an enzyme such as HRP or ALP or biotin.

31. A method according to claim 29, further comprising a step of contacting the immobilization matrix comprising the captured analyte with a substance capable of reacting with the biological marker.

32. A method according to claim 29, where the biological marker is one or more selected from mono-, oligo- and polyclonal antibodies, antigens, receptors, ligands, enzymes, proteins, peptides and nucleic acids.

33. A method according to claim 27, where the step f) is performed by moving a magnetic source along the external edge of the reaction chamber toward the detection part of the chamber.
